(19) United States
(12) Patent Application Publication
(10) Pub. No.: US 2018/0028186 A1
(43) Pub. Date: Feb. 1, 2018

(54) ORGAN ANASTOMOSIS SYSTEM

(71) Applicant: YUGENGAISHA PACS OPTICA JAPAN, Meguro-Ku (JP)

(72) Inventor: Eigoro YAMANOUCHI, Minato (JP)

(73) Assignee: YUGENGAISHA PACS OPTICA JAPAN, Meguro-Ku (JP)

(21) Appl. No.: 15/661,730

(22) Filed: Jul. 27, 2017

(30) Foreign Application Priority Data
Aug. 1, 2016 (JP) 2016-151302

(51) Int. Cl. A61B 17/11; A61B 17/17

(52) U.S. Cl. CPC A61B 17/1114; A61B 17/1796; A61B 2017/22038

(57) ABSTRACT

An organ anastomosis system 1 includes a pair of disc-shaped magnets 2 and 3 which pinch and compress adjacent organ walls 6a and 6b of an object by magnetically attracting each other with the organ walls 6a and 6b interposed therebetween so as to cause local apoptosis and form a continuous hole and an anastomotic part conglutinating around the peripheral part of the continuous hole are formed, which continuous hole specially connects the organ walls with each other. The side circumferential surfaces of respective disc-shaped magnetic bodies 2a and 3a of the pair of the magnets 2 and 3 are formed as tapered surfaces 2b and 3b which gradually decrease in diameter from one end to the other end in the axial direction of the magnetic bodies 2a and 3a.

### CROSS-REFERENCE TO RELATED APPLICATION

This application is based upon and claims the benefit of priority from Japanese Patent application No. 2016-151302, filed on Aug. 1, 2016, the entire contents of which are incorporated herein by reference.

### FIELD OF THE INVENTION

The present invention relates to an organ anastomosis system that locally causes apoptosis by strongly compressing (pinching) a pair of adjacent walls of one or plural internal organs of an object such as a human body each other making use of a magnetic attraction of a pair of magnets and then forms a bypass by forming a continuous hole and an anastomotic part conglutinating around the peripheral part of the continuous hole, which the continuous hole connects these organ walls with each other.

### RELATED ART

In general, when stenosis of an intestine or a bile duct of an object (e.g., a human body) is advanced due to, e.g., a tumor, an ulcer, inflammation, and/or external injury, anastomosis of the organ is often performed in order to, e.g., form a bypass (continuous hole) between two hollow intestine parts and to drain contents of the intestine and/or bile of the bile duct again by bypassing the stenosed part.

As an example of a conventional organ anastomosis system used for the anastomosis described above, an organ anastomosis system is known which uses a pair of magnets in disc shape. The present inventor has succeeded in many surgeries under the magnetic compression method by using a conventional organ anastomosis system as a surgeon over many years.

In a conventional organ anastomosis system as shown in FIG. 5A and FIG. 5B, a pair of magnets a and b are disc-shaped and the side circumferential surfaces a1 and b1 of the respective magnets a and b are flat.

### SUMMARY OF THE INVENTION

The present invention aims to provide an organ anastomosis system which can improve the probability of the successful forming of a continuous hole and an anastomotic part on an organ wall.

An organ anastomosis system of one embodiment is characterized in that tapered parts are formed on respective side circumferential surfaces of a pair of magnets.

### BRIEF DESCRIPTION OF THE DRAWINGS

FIG. 1 is an enlarged oblique view of an organ anastomosis system of one embodiment;

FIG. 2 is a partial longitudinal cross-sectional view illustrating a method for forming a bypass (i.e., a continuous hole and an anastomotic part) of a stenosed intestine by using a organ anastomosis system which is equipped with the pair of the magnets shown in FIG. 1;

FIG. 3 is an enlarged view of a continuous hole and an anastomotic part;

FIG. 4A is a schematic diagram illustrating a scene in which the tapered side circumferential surfaces of the pair of the magnets shown in FIG. 1 are placed inside a human body in the state of being in contact with a pair of adjacent organ walls;

FIG. 4B is a side view of the scene of FIG. 4A;

FIG. 4C is a schematic diagram illustrating a scene in which the pair of the magnets fall down on a pair of adjacent organ walls and the respective small diameter ends of the magnets are attracted to each other across the pair of the organ walls so as to pinch and compress the pair of the organ walls;

FIG. 4D is a schematic diagram illustrating a scene in which a pair of pinched organ walls locally cause apoptosis to form a continuous hole and an anastomotic part, the respective small diameter ends of the pair of the magnets being directly stuck to each other inside the continuous hole;

FIG. 4E is a schematic diagram illustrating a scene in which the pair of the magnets directly stuck to each other are dropping into inside of one of the organs (large intestine) from the continuous hole;

FIG. 5A is a schematic diagram illustrating a state where a pair of magnets of a conventional organ anastomosis system are erected at the upper side and the lower side of a pair of adjacent organ walls; and

FIG. 5B is a side view of the scene of FIG. 5A.

### DESCRIPTION OF THE EMBODIMENTS

Hereinbelow, embodiments of the present invention will be described with reference to the accompanying drawings. Note that the same reference signs are given for identical components or corresponding components in each figure.

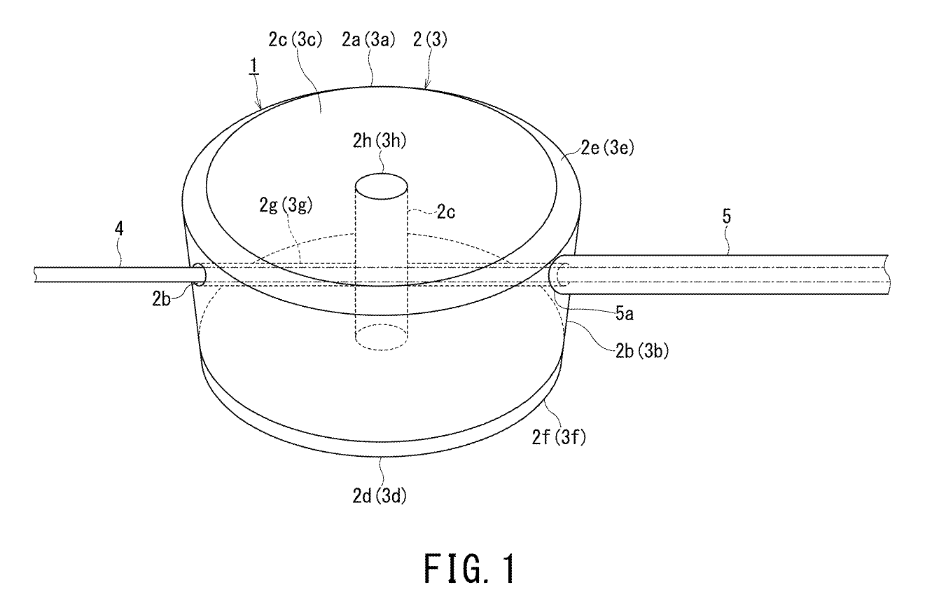

FIG. 1 is an oblique view of a pair of magnets 2 and 3 of an organ anastomosis system 1 according to the present embodiment.

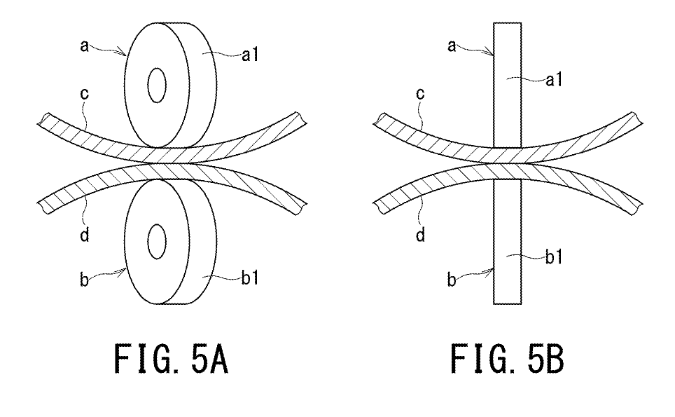

The conventional magnets a and b shown in FIG. 5B are formed to have flat side circumferential surfaces a1 and b1 as described above. Thus, when these magnets a and b are placed inside a target organ of an object and are arranged so as to face each other with a pair of adjacent walls of the organ interposed therebetween, there is a possibility that these magnets a and b stand upright under the state where their side circumferential surfaces a1 and b1 are in contact with the respective organ walls c and d.

Since the pair of the magnets a and b cannot be magnetically attracted to each other in this case, it is impossible to strongly pinch (press) the pair of the adjacent organ walls c and d each other by using the pair of the magnets a and b.

Hence, local apoptosis due to strong pinching (pressure) between these organ walls c and d does not occur, and there is a problem that a continuous hole and an anastomotic part cannot be formed on these organ walls c and d.

For this reason, the respective magnets 2 and 3 constituting the organ anastomosis system 1 of the present embodiment include disc-shaped magnet bodies 2a and 3a each of which are made of a magnet such as a rare earth element magnet and are substantially equal to each other in shape and size. Further, the respective side circumferential surfaces of the magnet bodies 2a and 3a have tapered surfaces 2b and 3b.

Each of the tapered surfaces 2b and 3b is formed by a tapered surface which gradually decreases in diameter from one end to the other end in the axial direction of the magnet body 2a or 3a (e.g., from the upper end to the lower end in FIG. 1). The taper angle is, e.g., 1 to 3 degrees.

In the magnet body 2a, a chamfered portion 2e is formed at the upper corner where the large diameter end 2c and the tapered surface 2b at the upper end in FIG. 1 intersect each other, and a chamfered portion 2f is formed at the lower corner where the large diameter end 2d and the tapered surface 2f at the lower end in FIG. 1 intersect each other. Similarly, in the magnet body 3a, a chamfered portion 3e is formed at the upper corner where the large diameter end 3c and the tapered surface 3b at the upper end in FIG. 1 intersect each other, and a chamfered portion 3f is formed at the lower corner where the large diameter end 3d and the tapered surface 3f at the lower end in FIG. 1 intersect each other.

The respective small diameter ends 2d and 3d of the pair of the magnets 2 and 3 are formed as magnetic poles (i.e., S pole and N pole) such that the magnetic polarity of the small diameter end 2d is opposite to that of the small diameter ends 3d. Further, the small diameter ends 2d and 3d are formed as attraction surfaces magnetically attracted to each other.

Additionally, almost all the outer surfaces of the magnet bodies 2a and 3a are coated with at least one of an acid-resistant film, a sulfur-resistant film, and an antithrombogenic film.

Further, in the magnet body 2a, a transverse hole 2g penetrating in the diametrical direction is formed. Similarly, in the magnet body 3a, a transverse hole 3g penetrating in the diametrical direction is formed. The transverse holes 2g and 3g are insertion holes through which a flexible guidewire 4 is inserted so as to be movable in the axial direction. The guide wire 4 is a wire that guides the respective magnet bodies 2a and 3a by moving (transporting) the respective magnet bodies 2a and 3a to a target organ wall of a human body, and is inserted from outside the body such as the nose, the mouth, and the anus of the human body.

Additionally, in the magnet body 2a, a vertical hole 2h penetrating in the axial direction is formed at the radial center portion of the magnet body 2a. Similarly, in the magnet body 3a, a vertical hole 3h penetrating in the axial direction is formed at the radial center portion of the magnet body 3a. The vertical holes 2h and 3h are insertion holes for inserting a collection cord which is for collecting the pair of the magnets 2 and 3 placed in a human body to the outside of the human body. A non-illustrated cross bar is provided on the side of one axial end of each of the vertical holes 2h and 3h (e.g., on the side of the small diameter end 2d and 3d), and horizontally extends in the diameter direction of each of the vertical holes 2h and 3h. Each of the vertical holes 2h and 3h is formed such that a non-illustrated hook provided at, e.g., a tip portion of the collection cord can be hooked on the cross bar.

Moreover, the organ anastomosis system 1 includes a guide tube 5 in the form of a circular pipe. The guide tube 5 includes a tip portion 5a to be brought into contact with the side circumferential surfaces of the pair of the magnets 2 and 3, and is formed into a circular tube shape with a material having substantially the same composition as that of an entirely flexible drainage tube.

An insertion hole through which the guide wire 4 is axially inserted with looseness is formed over the entire length of the axial center portion of the guide tube 5. The guide tube 5 is formed of, e.g., a soft vinyl chloride resin and/or a polyurethane resin in order to obtain rigidity necessary for appropriately imparting so-called pushability, torque transmissibility, and followability. A lubricant such as silicone oil may be contained in these resins in order to improve the slidability with the guide wire 4.

The above-described pushability means a characteristic that the force for pushing the guide tube 5 from the base end side (e.g., grasping side) to the side of the tip portion 5a can be reliably transmitted from the base end side to the side of the tip portion 5a when the force is applied by an operator in order to advance the guide tube 5 in an organ such as an intestinal tract and a blood vessel.

Additionally, the above-described torque transmissibility refers to a characteristic that the rotational force around the axis applied at the base end side of the guide tube 5 can be reliably transmitted to the tip side of the guide tube 5. Further, the above-described followability means a trackability that the guide tube 5 can smoothly and reliably advance along the guide wire 4 which precedes the inside of an organ such as a bent intestinal tract or a blood vessel.

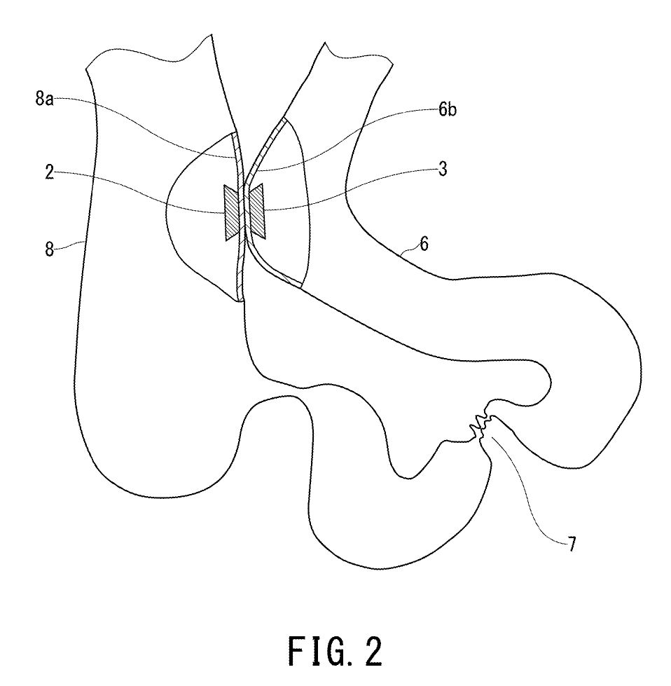

FIG. 2 is a partial longitudinal cross-sectional view of an organ around a continuous hole 9 (bypass) formed by anastomosis in the case of occurrence of an occlusion 7 (ileus) in the small intestine 6 such that the continuous hole 9 spatially connects the organ wall 6a of the target portion of the small intestine 6 on the upstream side of the occlusion 7 with the organ wall 8a of the target portion of the large intestine 8.

In this case, a surgeon inserts the tip portion of the guide wire 4 into the planned insertion path from, e.g., the nose or mouth of the human body while observing a non-illustrated X-ray fluoroscopic screen. Then, the tip portion of the guide wire 4 is advanced in the insertion path while meandering along the inner wall of the insertion path, and reaches the target organ wall 6a.

After this, the outer end portion of the guide wire 4 slightly extending outward from the nose or mouth is inserted into the transverse hole 2g of the first magnet 2 outside the nose or mouth, and the inserted tip portion is made to protrude outward from one end of the transverse hole 2g.

Afterward, a protuberant end portion of the guide wire 4 protruding outward from one end of the transverse hole 2g is inserted into the guide tube 5 as shown in FIG. 1, and the tip portion 5a of the guide tube 5 is brought into contact with the side circumferential surface 2b of the first magnet 2.

Next, the guide tube 5 is gradually pushed into inside of the human body from the outside of the nose or mouth of the human body.

Then, the first magnet 2 is pushed by the guide tube 5 and gradually slides on the guide wire 4 so as to be guided to the vicinity of the target organ wall 6a of the small intestine 6 as the destination.

At this timing, the first magnet 2 is further slightly pushed into inside of the human body by the guide tube 5, and the first magnet 2 is dropped from the guide wire 4. Thereby, the first magnet 2 is placed (indwelled) inside the target organ wall 6a which is the destination of the small intestine 6.

Thereafter, the guide wire 4 and the guide tube 5 are pulled back from the nose or mouth to the outside of the human body and are collected.

The second magnet 3 is transported to the vicinity of the organ wall 8a of the large intestine 8 on the downstream side of the stenosed portion 7 by the guide wire 4 and the guide tube 5 which have been inserted from, e.g., the anus side of the human body, and is placed. The method of inserting the guide wire 4 and the guide tube 5 from the anus side is substantially the same as the insertion method (procedure) of the guide wire 4 and the guide tube 5 inserted into the human body from the nose or mouth side except the difference in insertion position between both.

As described above, the pair of the magnets 2 and 3 are placed inside the body across the pair of the target organ wall 6a of the small intestine 6 and the target organ wall 8a of the large intestine 8, in such a manner that the small diameter ends 2d and 3d having different magnetic poles are arranged so as to face each other across the pair of the organ walls 6a and 8a. As the result, these small diameter ends 2d and 3d are strongly magnetically attracted to each other with the pair of the organ walls 6a and 8a interposed therebetween.

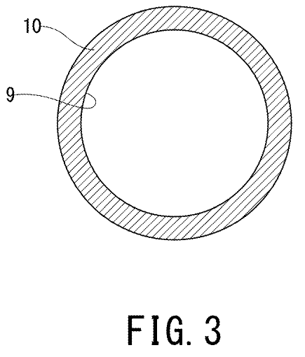

Accordingly, the pair of the organ walls 6a and 8a are strongly pinched and compressed by the pair of the magnets 2 and 3, and thus apoptosis occurs at this compressed part. Hence, the continuous hole 9 having substantially the same shape and the same size as the plane of each of the small diameter ends 2d and 3d of the pair of the magnets 2 and 3 is formed as shown in FIG. 3. Additionally, an annular anastomosis portion 10 is formed by conglutination on the outer peripheral edge portion of the continuous hole 9.

In other words, a bypass is formed in such a manner that the small intestine 6 and the large intestine 8 are connected to each other via the continuous hole 9 on the upstream side of the stenosed portion 7 of the small intestine 6.

However, there are some cases where the small diameter ends 2d and 3d of the pair of the magnets 2 and 3 cannot be arranged so as to face each other across the pair of the organ walls 6 a and 8a.

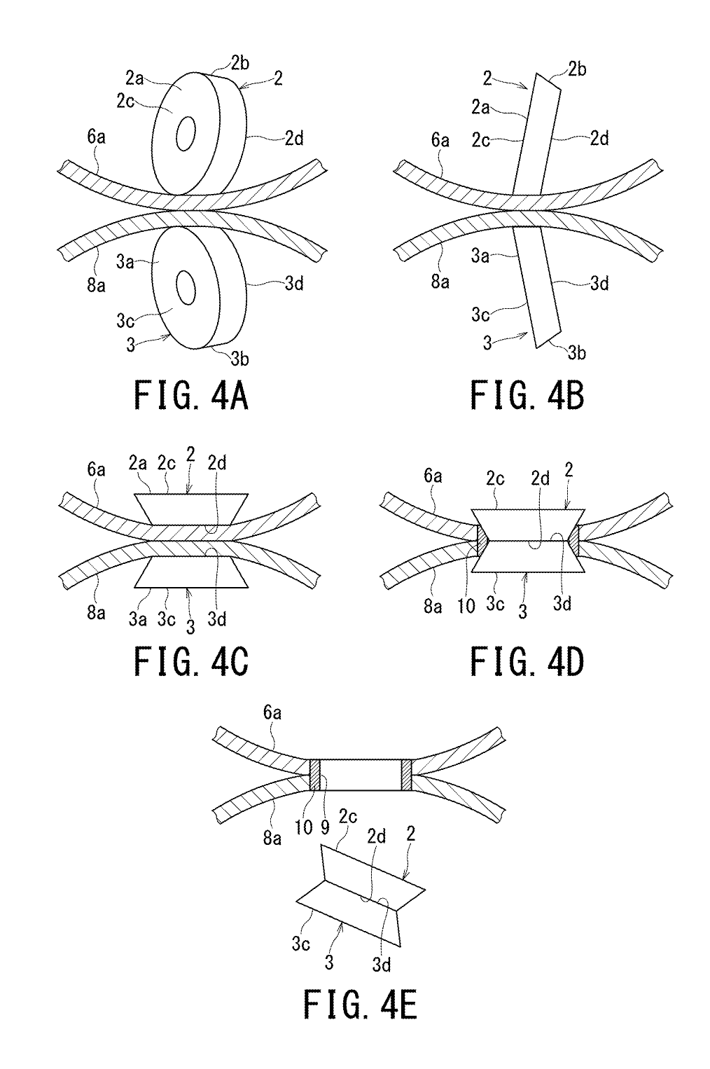

For instance, there are some cases where the pair of the magnets 2 and 3 are arranged inside the body such that not their small diameter ends 2d and 3d but their side circumferential surfaces 2b and 3b are in contact with the pair of the organ walls 6a and 8a as shown in FIG. 4A and FIG. 4B.

However, the side circumferential surfaces 2b and 3b of the respective magnets 2 and 3 are tapered so as to gradually reduce in diameter from the large diameter ends 2c and 3c toward the small diameter ends 2d and 3d in this case, and thus the pair of the magnets 2 and 3 naturally fall down such that the small diameter ends 2d and 3d face toward the pair of the organ walls 6a and 8a as shown in FIG. 4C.

Hence, the small diameter ends 2d and 3d of the pair of the magnets 2 and 3 are arranged so as to face each other across the pair of the organ walls 6a and 8a, are strongly magnetically attracted to each other, and strongly pinches and compresses the pair of the organ walls 6a and 8a.

Thus, this compressed part causes apoptosis. Accordingly, the continuous hole 9 and the anastomosis portion 10 conglutinating at the outer peripheral edge of the continuous hole 9 are formed between the small diameter ends 2d and 3d of the pair of the magnets 2 and 3, in such a manner that the continuous hole 9 becomes circular and has substantially the same shape and the same size as the planar shape of the small diameter ends 2d and 3d as shown in FIG. 4D. As the result, the small diameter ends 2d and 3d of the pair of the magnets 2 and 3 are directly stuck to each other in the continuous hole 9.

Thereafter, after elapse of a required time, the pair of the magnets 2 and 3 are pushed out from the continuous hole 9 by the internal pressure of the intestine and the own weight of the magnets 2 and 3 and fall to one organ wall side (e.g., the side of the large intestine 8 inside the organ wall 8a), under the state where the small diameter ends 2d and 3d are directly stuck to each other as shown in FIG. 4E.

Although the large diameter ends 2c and 3c of the pair of the magnets 2 and 3 are slightly larger than the small diameter ends 2d and 3d by the taper angle and are slightly larger in diameter than the continuous hole 9, the large diameter ends 2c and 3c are formed in such a size that they can pass through the continuous hole 9 due to the taper angle. Moreover, mucous membranes in and around the continuous hole 9 have slippage due to various body fluids. Thus, the pair of the magnets 2 and 3 slide in the continuous hole 9 and fall into the large intestine 8 inside one organ wall (e.g., the organ wall 8a), under the state where the small diameter ends 2d and 3d are attracted to each other. Finally, the pair of the magnets 2 and 3 are discharged outside the body by defecation.

Hence, according to the organ anastomosis system 1, even when the pair of the magnets 2 and 3 are placed inside a human body in the so-called standing state where their side circumferential surfaces 2b and 3b are in contact with the pair of the organ walls 6a and 8a, the side circumferential surfaces 2b and 3b are tapered so as to be inclined at required angles and thus it is possible to magnetically attract the small diameter ends 2d and 3d to each other by making the upright magnets 2 and 3 fall on the pair of the organ walls 6a and 8a. As the result, it is possible to increase the probability that the continuous hole 9 and the anastomosis portion 10 are successfully formed.

Furthermore, the organ anastomosis system 1 has a simple configuration in which the side circumferential surfaces of the pair of the magnets 2 and 3 are formed as the tapered surfaces 2b and 3b, and thus it is possible to suppress manufacturing cost.

Although a method of using the guide wire 4 and the guide tube 5 has been described as a method of disposing the second magnet 3 on the side of the large intestine 8 in a manner similar to the method of disposing the first magnet 2 on the side of the small intestine 6 in the above embodiment, the present invention is not limited to such an aspect.

For instance, the second magnet 3 may be grasped by a non-magnetic grasping forceps of an endoscope and be inserted into inside of the large intestine 8 from the anus so as to be left placed.

Additionally, as a method of recovering the magnet 3 out of the human body, the tip portion of the collection cord may be inserted from, e.g., the anus of the human body and be inserted into the vertical hole 3h of the magnet 3, while the surgeon is observing an X-ray fluoroscopic screen. In this case, the magnet 3 is collected by hooking the hook of the tip portion to the cross bar and pulling back the collection cord from the exterior (e.g., from the anus).

Further, though a description has been given of the method in which the organ-anastomotic apparatus 1 is used for enterocleisis (ileus) in the above embodiment, the present invention is not limited to such an aspect and can also be applied to, e.g., common bile-duct stenosis. In this case, one of the pair of the magnets 2 and 3 may be inserted from the percutaneous transhepatic bile-duct drainage-route and the other of the magnets 2 and 3 may be grasped by the grasping forceps of the endoscope and be inserted into the intestinal tract such as the duodenum so as to be placed.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. An organ anastomosis system comprising a pair of disc-shaped magnets which pinch adjacent organ walls of an object by magnetically attracting each other across the organ walls to cause local apoptosis and form a continuous hole and an anastomotic part conglutinating around a peripheral part of the continuous hole, the continuous hole specially connecting the organ walls with each other,
wherein a side circumferential surface of a disc-shaped magnet body of each of the pair of disc-shaped magnets is formed as a tapered surface which gradually decreases in diameter from one end to another end in an axial direction of the magnet body.

2. The organ anastomosis system according to claim 1,
wherein respective small diameter ends of disc-shaped magnet bodies of the pair of disc-shaped magnets are formed as magnetic poles in such a manner that respective magnetic polarities of the small diameter ends are opposite to each other.

3. The organ anastomosis system according to claim 1,
wherein a large diameter end of the disc-shaped magnet body of each of the pair of disc-shaped magnets is formed in such a size that the large diameter end can pass through the continuous hole due.

4. The organ anastomosis system according to claim 1,
wherein a transverse hole is formed on at least one of the pair of disc-shaped magnets, in such a manner that the transverse hole penetrates the disc-shaped magnet body in a diametrical direction of the disc-shaped magnet body and a guide wire can be inserted into the transverse hole in a state of being movable in a longitudinal direction.

5. The organ anastomosis system according to claim 1, further comprising:
an insertion hole into which a guide wire can be inserted in a state of being movable in a longitudinal direction;
a tip portion configured to be brought into contact with the side circumferential surface under a state where the guide wire is inserted into the insertion hole; and
a tube configured to move the pair of disc-shaped magnets by pressing the tip portion.

6. The organ anastomosis system according to claim 1,
wherein a vertical hole is formed on the disc-shaped magnet body of each of the pair of disc-shaped magnets in such a manner that the vertical hole penetrates through the disc-shaped magnet body in an axial direction and a cord for collecting the pair of disc-shaped magnets can be hooked to the vertical hole.
